(19) United States
(12) Patent Application Publication
(10) Pub. No.: US 2023/0192936 A1
(43) Pub. Date: Jun. 22, 2023

(54) TWO-COMPONENT POLYURETHANE COMPOSITION, AND PREPARATION METHOD AND APPLICATION THEREOF

(71) Applicant: Ningbo Baoting Biotechnology Co. , Ltd., Ningbo (CN)

(72) Inventors: Yabin Zhu, Ningbo (CN); Bin He, Ningbo (CN); Yun Bai, Ningbo (CN); Dongmei Huang, Ningbo (CN)

(21) Appl. No.: 17/990,965

(22) Filed: Nov. 21, 2022

Related U.S. Application Data
(63) Continuation of application No. PCT/CN2021/113561, filed on Aug. 19, 2021.

(30) Foreign Application Priority Data
Jun. 2, 2021 (CN) 2021106122182

(51) Int. Cl. C08G 18/12; C08G 18/48; C08G 18/32; C08G 18/16; C08K 5/11; C08K 5/17; C08L 75/08

(52) U.S. Cl. CPC C08G 18/12; C08G 18/4854; C08G 18/4895; C08G 18/4804; C08G 18/3206; C08G 18/16; C08K 5/11; C08K 5/17; C08L 75/08; C08L 2203/02

(57) ABSTRACT

The present invention provides a two-component polyurethane composition, and belongs to the technical field of high polymer material. The two-component polyurethane composition comprises component A and component B. According to mass percent, the component A comprises 0-100% of high activity polyether polyol, 0-100% of cross-linking agent, 0-40% of chain extender, 0-50% of plasticizer and 0-5% of catalyst; according to mass percent, the component B comprises 0-100% of diphenylmethane diisocyanate and derivative thereof, and 0-50% of plasticizer; and the mass ratio of the component A to the component B is 1:0.1-1:3. The two-component polyurethane composition provided by the present invention has low heat release and excellent mechanical properties.

### TECHNICAL FIELD

The present invention relates to the technical field of high polymer material, and in particular to a two-component polyurethane composition, and a preparation method and application thereof.

### BACKGROUND

A fracture fixation splint is a frequently-used auxiliary device in orthopedic treatment. At present, plaster and splints are used clinically to treat patients with bone injury. Traditional plaster and splint fixation modes have poor air permeability and high hardness. In the process of use, itching, friction pain, dermatitis and other problems will occur due to stuffiness, and the skin is squeezed due to high hardness, resulting in the problems of cutaneous necrosis, long-term inflammation and the like and making patients feel bad. In addition, when fracture occurs, the first reaction is that the fracture swells severely and the swelling is gradually decreased in the healing process. High-hardness gypsum boards or splints have no regulating function, and can only be replaced with new dressing, which wastes resources and increases the economic burden of the patients.

In addition to the traditional plaster fixation mode, polymer splints are used clinically for fixing the affected part. A bandage coated with water-based single-component polyurethane material is frequently used. In the process of use, the bandage needs to be soaked in water, twisted to be dry, shaped, and applied to the patient for shaping. Its characteristics are light weight, high hardness and no elasticity, and has the problems of cumbersome use, poor air permeability, poor body sensation for the patients, stuffiness and itching.

### SUMMARY

In view of this, a first purpose of the present invention is to solve the deficiencies existing in the above background. Thus, the present invention provides a two-component polyurethane composition with low heat release and excellent mechanical properties.

To achieve the above purpose, the present invention provides the following technical solution:

A two-component polyurethane composition comprises component A and component B;

According to mass percent, the component A comprises 0-100% of high activity polyether polyol, 0-100% of cross-linking agent, 0-40% of chain extender, 0-50% of plasticizer and 0-5% of catalyst;

According to mass percent, the component B comprises 0-100% of diphenylmethane diisocyanate and derivative thereof, and 0-50% of plasticizer;

The mass ratio of the component A to the component B is 1:0.1-1:3.

Preferably, the polyether polyol is one of polyoxytrimethylene polyol, polytetrahydrofuran polyol and polyepoxide butane polyol.

Preferably, the cross-linking agent comprises at least one of a macromolecular cross-linking agent and a micromolecular cross-linking agent.

Preferably, the macromolecular cross-linking agent is high-functionality polyether polyol, preferably one of MN500, MN700 and MN1000.

Preferably, the micromolecular cross-linking agent is polyfunctionality micromolecular polyol, preferably at least one of tetrahydroxypropyl ethylenediamine, pentaerythritol, trimethylolpropane, glycerol and tetrahydroxypropyl ethylenediamine.

Preferably, the chain extender is micromolecular diol, preferably at least one of diethylene glycol, ethylene glycol and dipropylene glycol.

Preferably, the catalyst is an amine catalyst or a metallic salt catalyst.

Preferably, plasticizers in the component A and the component B comprise at least one of di-isononyl-cyclohexane-1,2-dicarboxylate, triethyl citrate, dibutyl phthalate, dioctyl sebacate, acetyl tributyl citrate, 2, 2, 4-trimethyl-1, 3-glutaryl diisobutyrate.

A second purpose of the present invention is to provide a preparation method for the two-component polyurethane composition, comprising the following steps:

preparation of the component A

respectively adding the measured component A into a dispersion machine, stirring to obtain viscous liquid, drying, and stirring for later use;

preparation of the component B

adding the measured diphenylmethane diisocyanate, the derivative thereof and the dried plasticizers into the dispersion machine, and stirring to form viscous liquid for later use;

preparation of the two-component polyurethane composition

respectively packing the prepared component A and component B into aluminum foil bags for later use;

pouring the component B into the aluminum foil bag of the component A, shaking and mixing evenly, and then squeezing into a matched silica gel bag to obtain the two-component polyurethane composition.

Preferably, an inner cavity of the silica gel bag is covered with an inner bag so that the silica gel bag has the function of anti-cracking.

A third purpose of the present invention is to provide the application of the two-component polyurethane composition in orthopedic treatment.

Compared with the prior art, the present invention has the following beneficial effects:

1. The two-component polyurethane composition provided by the present invention has low heat release and excellent mechanical properties.

2. The two-component polyurethane composition provided by the present invention can be applied to auxiliary equipment frequently used in orthopedic treatment, such as polymer splint. Because the two-component polyurethane composition has elastic and soft silicone surface and good skin affinity of the body, the composition can be arranged into cavities of various shapes as required, which can reduce the problems of the stuffiness, itching and poor air permeability of patients; and the two-component polyurethane composition has the advantages of elasticity, and capability of properly adjusting tightness to increase the comfort of the patients.

### DETAILED DESCRIPTION

To make purposes, technical solutions and advantages of the implementation modes of the present invention more clear, the technical solutions in the implementation modes of the present invention will be clearly and fully described below. Apparently, the described implementation modes are merely part of the implementation modes of the present invention, not all of the implementation modes. Based on the implementation modes in the present invention, all other implementation modes obtained by those ordinary skilled in the art without contributing creative labor will belong to the protection scope of the present invention. Therefore, the detailed description of the implementation modes of the present invention is not intended to limit the scope of the claimed present invention, and merely represents the selected implementation modes of the present invention.

A two-component polyurethane composition comprises component A and component B;

According to mass percent, the component A comprises 0-100% of high activity polyether polyol, 0-100% of cross-linking agent, 0-40% of chain extender, 0-50% of plasticizer and 0-5% of catalyst;

According to mass percent, the component B comprises 0-100% of diphenylmethane diisocyanate and derivative thereof, and 0-50% of plasticizer;

The mass ratio of the component A to the component B is 1:0.1-1:3.

Polyether polyol (polyether for short) is an organic polymer, which is prepared by the ring opening reaction of an initiator (compound containing active hydrogen group) with ethylene oxide (EO), propylene oxide (PO), butane oxide (BO) and the like under the action of a catalyst.

In the present invention, the polyether polyol is one of polyoxytrimethylene polyol, polytetrahydrofuran polyol and polyepoxide butane polyol.

In the present invention, the cross-linking agent comprises at least one of a macromolecular cross-linking agent and a micromolecular cross-linking agent. Because the micromolecular cross-linking agent causes hard and brittle products and the product prepared by the macromolecular cross-linking agent has certain toughness, the following embodiments in the present invention exemplarily show the simultaneous addition of the macromolecular cross-linking agent and the micromolecular cross-linking agent.

In the present invention, the macromolecular cross-linking agent is high-functionality polyether polyol, preferably one of MN500, MN700 and MN1000, which can be selected by those skilled in the art according to actual needs.

In the present invention, the micromolecular cross-linking agent is polyfunctionality micromolecular polyol, preferably at least one of tetrahydroxypropyl ethylenediamine, pentaerythritol, trimethylolpropane, glycerol and tetrahydroxypropyl ethylenediamine, which can be selected by those skilled in the art according to actual needs.

In the present invention, the chain extender is micromolecular diol, preferably at least one of diethylene glycol, ethylene glycol and dipropylene glycol, which can be selected by those skilled in the art according to actual needs.

Preferably, the catalyst is an amine catalyst or a metallic salt catalyst, which can be selected by those skilled in the art according to actual needs.

In the present invention, plasticizers in the component A and the component B comprise at least one of di-isononyl-cyclohexane-1,2-dicarboxylate, triethyl citrate, dibutyl phthalate, dioctyl sebacate, acetyl tributyl citrate, 2, 2, 4-trimethyl-1, 3-glutaryl diisobutyrate.

The present invention provides a preparation method for the two-component polyurethane composition, comprising the following steps:

adding the measured component A to a high-speed dispersion machine respectively; stirring at high speed for 0.5-6 hours to form viscous liquid with viscosity of 200-6000 mPa s/25° C.; adding a zeolite of 2-30% mass part; and stirring at low speed for 72 hours for later use, or dehydrating in a high temperature dehydration mode for later use.

A preparation method for the component B comprises the following steps:

adding a zeolite of 2-30% mass part to the plasticizer; and stirring at low speed for 72 hours for later use. Adding the measured isocyanate and the dried plasticizer to the high-speed dispersion machine; and stirring at high speed for 0.5-3 hours to form viscous liquid with viscosity of 50-500 mPa s/25 for later use.

Preparation of the two-component polyurethane composition

respectively packing the prepared component A and component B into aluminum foil bags for later use;

pouring the component B into the aluminum foil bag of the component A, then squeezing into a matched silica gel bag after shaking and mixing evenly for 1 minute, and curing for about 10 minutes to obtain the two-component polyurethane composition.

Preferably, an inner cavity of the silica gel bag is covered with an inner bag so that the silica gel bag has the function of anti-cracking, to prevent the burst or damage during the perfusion of A\B glue from making the inner liquid overflow.

The present invention further provides the application of the two-component polyurethane composition in orthopedic treatment. For example, the two-component polyurethane composition is used for preparing an auxiliary device in orthopedic treatment, and for example, preparing a fracture fixation plate described in the background; and a hole is formed in the surface of the fixation plate. Because the two-component polyurethane composition has elastic and soft silicone surface and good skin affinity of the body, the composition can be arranged into cavities of various shapes as required, which can reduce the problems of the stuffiness, itching and poor air permeability of patients; and the two-component polyurethane composition has the advantages of elasticity, and capability of properly adjusting tightness to increase the comfort of the patients. More importantly, a doctor can observe the situation of bone injury by eyes through the cavity, and can also check the healing of an affected part through X-ray, CT and other instruments, which provides convenience for the treatment solution of the doctor.

The present invention will be further described in detail below in combination with specific embodiments.

Designations are described as follows. The following designations are exemplarily selected for chemical components in the present invention.

| 330N | Polyfunctionality soft foam polyether |
|---|---|
| DV125 | Polyfunctionality macromolecular cross-linking agent |
| MN500 | Polyfunctionality macromolecular cross-linking agent |
| MN700 | Polyfunctionality macromolecular cross-linking agent |
| MN1000 | Polyfunctionality macromolecular cross-linking agent |
| EDTP | Polyfunctionality micromolecular cross-linking agent |
| EG | Polyfunctionality micromolecular cross-linking agent |
| TMP | Polyfunctionality micromolecular cross-linking agent |
| TIPA | Polyfunctionality micromolecular cross-linking agent |
| DEG | Chain extender |
| TXIB | Plasticizer |
| PAPI | Polymethylene polyphenyl polyisocyanate (coarse MDI) |
| Liquefied MDI | Diphenylmethane diisocyanate derivative |
| A33 | Amine catalyst |
| 4110 | Metal salt catalyst |

### Embodiment 1

| Composition | Name | Mass part |
|---|---|---|
| Component | 330N | 30% |
| A | DV125 | 15% |
| | EDTP | 20% |
| | DPG | 5% |
| | TXIB | 30% |
| Component | Liquefied MDI | 30% |
| B | PAPI | 30% |
| | TXIB | 40% |

The component A and the component B in embodiment 1 are respectively packed in the aluminum foil bags according to the mass ratio of 1:1.2 and placed in a dry and shade environment for later use.

Use method: during use, the component B is poured into the aluminum foil bag of the component A, squeezed into a matched silica gel bag after shaking and mixing evenly for 1 minute, and cured for about 10 minutes.

Main technical indexes are shown in the following table:

| No. | Item | Unit | Index |
|---|---|---|---|
| 1 | appearance | mPa · s/25° C. | component A: 680 |
| | | | component B: 260 |
| 2 | density | g/cm³ | 1.12 |
| 3 | hardness | Shore D | 75 |
| 4 | gelation time | min | 2 |
| 5 | curing time | min | 10 |
| 6 | peak temperature | ° C. | 55 |

Embodiments 2-7 are shown in the following table:

| | | Embodiment | Embodiment | Embodiment | Embodiment | Embodiment | Embodiment |
|---|---|---|---|---|---|---|---|
| Composition | Name | 2 | 3 | 4 | 5 | 6 | 7 |
| Component | 330N | 20% | 40% | 60% | 80% | 30% | 40% |
| A | DV125 | 25% | | | | 15% | 15% |
| | MN500 | | 10% | | | | |
| | MN700 | | | 10% | | | |
| | MN1000 | | | | 5% | | |
| | EDTP | 20% | 15% | | | | |
| | EG | | | 10% | 4.98% | | |
| | TMP | | | | | 20% | |
| | TIPA | | | | | | 15% |
| | DEG | 4.98% | 4.98% | 4.98% | | 4.98% | 4.98% |
| | A33 | 0.02% | | 0.02% | 0.02% | | 0.02% |
| | 4110 | | 0.02% | | | 0.02% | |
| | TXIB | 30% | 30% | 15% | 10% | 30% | 25% |
| Component | PAPI | 25% | 25% | 30% | 30% | 30% | 30% |
| B | TXIB | 50% | 50% | 40% | 40% | 40% | 40% |
| | Liquefied | 25% | 25% | 30% | 30% | 30% | 30% |
| | MDI | | | | | | |
| Mass ratio | | 1:1.68 | 1:1.11 | 1:1.24 | 1:0.58 | 1:1.69 | 1:1.19 |
| Item | Unit | Formula 2 | Formula 3 | Formula 4 | Formula 5 | Formula 6 | Formula 7 |
| appearance | mPa · s/25° C. | A: 680 | A: 750 | A: 800 | A: 760 | A: 780 | A: 770 |
| | | B: 230 | B: 230 | B: 260 | B: 260 | B: 230 | B: 260 |
| density | g/cm³ | 1.18 | 1.18 | 1.12 | 1.16 | 1.16 | 1.16 |
| hardness | Shore D | 70 | 68 | 75 | 72 | 74 | 72 |
| Gelation time | min | 2.5 | 2.8 | 2 | 2.5 | 2.5 | 2.5 |
| Curing time | min | 13 | 13 | 11 | 13 | 13 | 15 |
| Peak temperature | ° C. | 55 | 55 | 57 | 60 | 58 | 52 |

1. A two-component polyurethane composition, comprising component A and component B, wherein
according to mass percent, the component A comprises 0-100% of high activity polyether polyol, 0-100% of cross-linking agent, 0-40% of chain extender, 0-50% of plasticizer and 0-5% of catalyst;
according to mass percent, the component B comprises 0-100% of diphenylmethane diisocyanate and derivative thereof, and 0-50% of plasticizer;
the mass ratio of the component A to the component B is 1:0.1-1:3.

2. The two-component polyurethane composition according to claim 1, wherein the polyether polyol is one of polyoxytrimethylene polyol, polytetrahydrofuran polyol and polyepoxide butane polyol.

3. The two-component polyurethane composition according to claim 1, wherein the cross-linking agent comprises at least one of a macromolecular cross-linking agent and a micromolecular cross-linking agent.

4. The two-component polyurethane composition according to claim 3, wherein the macromolecular cross-linking agent is high-functionality polyether polyol, preferably one of MN500, MN700 and MN1000.

5. The two-component polyurethane composition according to claim 3, wherein the micromolecular cross-linking agent is polyfunctionality micromolecular polyol, preferably at least one of tetrahydroxypropyl ethylenediamine, pentaerythritol, trimethylolpropane, glycerol and tetrahydroxypropyl ethylenediamine.

6. The two-component polyurethane composition according to claim 1, wherein the chain extender is micromolecular diol, preferably at least one of diethylene glycol, ethylene glycol and dipropylene glycol.

7. The two-component polyurethane composition according to claim 1, wherein the catalyst is an amine catalyst or a metallic salt catalyst.

8. The two-component polyurethane composition according to claim 1, wherein plasticizers in the component A and the component B comprise at least one of di-isononyl-cyclohexane-1,2-dicarboxylate, triethyl citrate, dibutyl phthalate, dioctyl sebacate, acetyl tributyl citrate, 2, 2, 4-trimethyl-1, 3-glutaryl diisobutyrate.

9. (canceled)

10. (canceled)

11. A preparation method for the two-component polyurethane composition of claim 1, comprising the following steps:
preparation of the component A
respectively adding the measured component A into a dispersion machine, stirring to obtain viscous liquid, drying, and stirring for later use;
preparation of the component B
adding the measured diphenylmethane diisocyanate, the derivative thereof and the dried plasticizers into the dispersion machine, and stirring to form viscous liquid for later use;
preparation of the two-component polyurethane composition
respectively packing the prepared component A and component B into aluminum foil bags for later use;
pouring the component B into the aluminum foil bag of the component A, shaking and mixing evenly, and then squeezing into a matched silica gel bag to obtain the two-component polyurethane composition;
preferably, an inner cavity of the silica gel bag is covered with an inner bag so that the silica gel bag has the function of anti-cracking.

12. An application of the two-component polyurethane composition of claim 1 in orthopedic treatment.
